(19) United States
(12) Patent Application Publication
(10) Pub. No.: US 2007/0250114 A1
(43) Pub. Date: Oct. 25, 2007

(54) Flexible tissue sheath for fibrous connective tissue repair

(75) Inventor: Susan J. Drapeau, Cordova, TN (US)

Correspondence Address: LARSON NEWMAN ABEL POLANSKY & WHITE, LLP, 5914 WEST COURTYARD DRIVE, SUITE 200, AUSTIN, TX, 78730, US

(73) Assignee: SDGI HOLDINGS, INC., Wilmington, DE (US)

(21) Appl. No.: 11/407,615

(22) Filed: Apr. 20, 2006

(51) Int. Cl. A61B 17/08

(52) U.S. Cl. 606/215; 606/152

(57) ABSTRACT

A flexible tissue sheath for treating a lacerated fibrous connective tissue is disclosed and can include a hollow body that can define an interior surface and an exterior surface. At least one tissue engagement structure can extend from the hollow body. The tissue engagement structure can engage an end of the lacerated fibrous connective tissue and substantially prevent the end of the lacerated fibrous connective tissue from withdrawing from the hollow body.

### FIELD OF THE DISCLOSURE

The present disclosure relates generally to orthopedics and orthopedic surgery. More specifically, the present disclosure relates to devices used to repair injured ligaments or injured tendons.

### BACKGROUND

An adult human skeleton includes two hundred and six bones. Further, an adult human includes approximately six hundred and fifty muscles. Numerous ligaments within the body connect bone to bone. Also, numerous tendons within the body connect muscles to bone.

Ligaments and tendons are fibrous connective tissue. During a lifetime, a human may injure one or more ligaments or tendons. For example, a tendon or ligament may be lacerated due to injury and may require repair. The ends of the tendon, or ligament, may be brought together and sutured to each other. During treatment, it can be advantageous to deliver a therapeutic agent to a lacerated tendon or ligament.

### BRIEF DESCRIPTION OF THE DRAWINGS

FIG. 1 is a view of a flexible tissue sheath;

FIG. 2 is a cross-section view of the flexible tissue sheath;

FIG. 3 is a view of the flexible tissue sheath installed around a tendon;

FIG. 4 is flow chart illustrating a method of repairing a torn tendon;

FIG. 5 is a plan view of a second flexible tissue sheath;

FIG. 6 is a cross-section view of the second flexible tissue sheath;

FIG. 7 is flow chart illustrating a method of repairing an injured tendon;

FIG. 8 is a plan view of a third flexible tissue sheath;

FIG. 9 is a cross-section view of the third flexible tissue sheath

FIG. 10 is a plan view of a fourth flexible tissue sheath;

FIG. 11 is a cross-section view of the fourth flexible tissue sheath; and

FIG. 12 is flow chart illustrating a second method of repairing a torn tendon.

### DETAILED DESCRIPTION OF THE DRAWINGS

A flexible tissue sheath for treating a lacerated fibrous connective tissue is disclosed and can include a hollow body that can define an interior surface and an exterior surface. At least one tissue engagement structure can extend from the hollow body. The tissue engagement structure can engage an end of the lacerated fibrous connective tissue and substantially prevent the end of the lacerated fibrous connective tissue from withdrawing from the hollow body.

In another embodiment, a method of treating a lacerated fibrous connective tissue is disclosed and can include inserting a first end of the lacerated fibrous connective tissue into a first end of a flexible tissue sheath so that the first end of the lacerated fibrous connective tissue engages a first set of tissue engagement structures.

In yet another embodiment, a flexible tissue sheath for treating an injured fibrous connective tissue is disclosed and can include a generally flat body configured to be wrapped around the injured fibrous connective tissue. The body can include a tissue engagement portion that can engage a portion of the injured fibrous connective tissue when the flexible tissue sheath is wrapped around the injured fibrous connective tissue.

In still another embodiment, a method of treating an injured fibrous connective tissue is disclosed and can include positioning a flexible tissue sheath proximate to an injured area of the fibrous connective tissue. The flexible tissue sheath can include a tissue engagement structure. Further, the method can include wrapping the flexible tissue sheath around the fibrous connective tissue so that the tissue engagement structure engages the fibrous connective tissue proximate to the injured area.

In another embodiment, a method of treating a lacerated fibrous connective tissue is disclosed and can include inserting a first end of the lacerated fibrous connective tissue into a first end of a flexible tissue sheath so that the first end of the lacerated fibrous connective tissue engages a first set of tissue engagement structures. The method can also include tightening the first end of the flexible tissue sheath around the first end of the lacerated fibrous connective tissue.

### Description of a First Embodiment of a Flexible Tissue Sheath

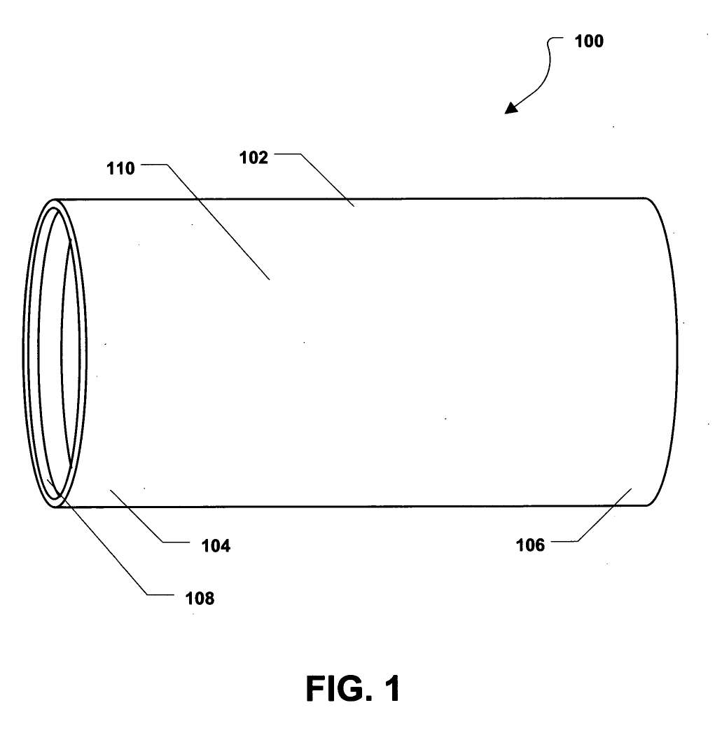

Referring to FIG. 1, a flexible tissue sheath is shown and is generally designated 100. As illustrated, the flexible tissue sheath 100 includes a body 102. In a particular embodiment, the body 102 can be generally hollow and generally cylindrical. Further, the body 102 can be flexible. Also, the body 102 can have a tensile strength that can allow the flexible tissue sheath 100 to function in unison with fibrous connective tissue, e.g., a tendon or ligament, around which the flexible tissue sheath 100 can be installed, as described herein.

In a particular embodiment, body 102 of the flexible tissue sheath 100 can be made from a biocompatible material. Further, the biocompatible material can include a natural polymer, a synthetic polymer, a blend of a natural polymer and a synthetic polymer, or a combination thereof. The natural polymer can include collagen.

The synthetic polymers can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

The polymer blends can include a blend of collagen and one or more of the above synthetic polymers. Further, the polymer blends can include a blend of collagen and alginate, a blend of collagen and polyethylglycol (PEG), a blend of collagen and polylactone, or a combination thereof.

In a particular embodiment, the body 102 can define a first end 104 and a second end 106 that is opposite the first end 104. The body 102 can also include an interior surface 108 and an exterior surface 110.

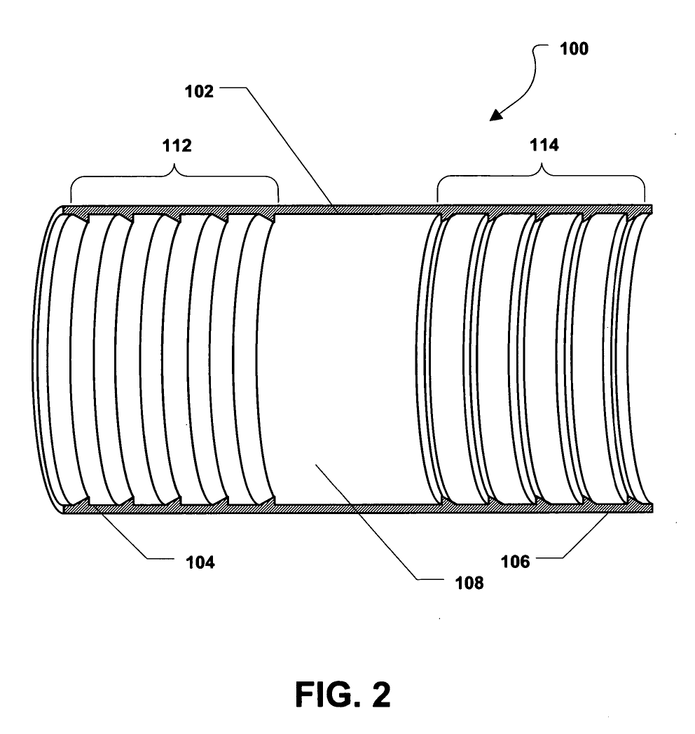

As depicted in FIG. 2, the interior surface 108 can include a first set of directional tissue engagement structures 112 that can extend from the interior surface 108 between a center of the body 102 and the first end 104 of the body 102. Further, the interior surface 108 can include a second set of directional tissue engagement structures 114 that can extend from the interior surface 108 between the center of the body 102 and the second end of 106 the body 102.

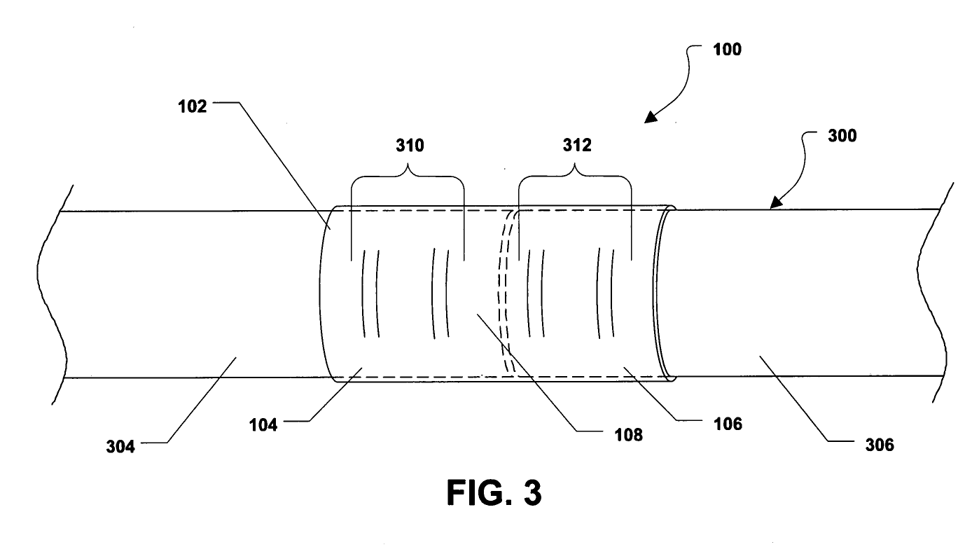

In a particular embodiment, the directional tissue engagement structures 112, 114 can be configured to engage tissue, e.g., a tendon or a ligament, in a single direction. Accordingly, the flexible tissue sheath 100 can be inserted over an end of a tendon, as shown in FIG. 3 and described in detail below, without the directional tissue engagement structures 112, 114 interfering with the insertion of the flexible tissue sheath 100 over the end of the tendon. However, after the flexible tissue sheath 100 is inserted over the end of the tendon, the directional tissue engagement structures 112, 114 can engage the end of the tendon and substantially reduce the ease of removing the flexible tissue sheath 100 from the end of the tendon. Accordingly, it can be relatively easy to slide, or otherwise install, the flexible tissue sheath 100 over an end of a tendon and it can be relatively difficult to remove the flexible tissue sheath 100 from the end of the tendon.

In a particular embodiment, as illustrated in FIG. 2, the directional tissue engagement structures 112, 114 can be ramped structures, barbs, teeth, spikes, a combination thereof, or some other structure designed to allow tissue to slide relative to the directional tissue engagement structures 112 in a single direction. Further, in a particular embodiment, the first set of directional tissue engagement structures 112 can be oriented opposite the second set of directional tissue engagement structures 114.

In other words, each set of directional tissue engagement structures 112, 114 can each be oriented to allow a tendon end to be slid, or otherwise moved, into the flexible tissue sheath 100 from opposite ends 104, 106 of the sheath 100. As such, two ends of a lacerated tendon can be inserted into the flexible tissue sheath 100 and the flexible tissue sheath 100 can substantially prevent the ends of the lacerated tendon from being removed from the flexible tissue sheath 100. Accordingly, the flexible tissue sheath 100 can act as a coupling mechanism in order to couple the ends of a lacerated tendon together to allow the ends of the lacerated tendon to heal and the tendon to repair itself.

In various alternative embodiments, one or both sets of directional tissue engagement structures 112, 114 can include structures of various sizes. For example, the height of the structures can increase or decrease from the center of the body 102 to either or both ends 104, 106, thereby defining a tapered aperture through which the ligament or tendon is inserted or moved. Similarly, one or both sets of directional tissue engagement structures 112, 114 can vary in shape at one or more points along the longitudinal axis of the body 102 in order to accommodate tissue of various shapes and conditions.

As illustrated in FIG. 1 and FIG. 3, the exterior surface 110 of the body 102 can be substantially smooth. Further, the exterior surface 110 of the body 102 can substantially prevent tissue adhesion to the exterior surface 110 of the body 102. Also, the exterior surface 110 can substantially minimize scarring around the lacerated tendon and the flexible tissue sheath 100 used to repair the lacerated tendon.

In a particular embodiment, the body 102 of the flexible tissue sheath 100 can be porous. As such, the flexible tissue sheath 100 can be loaded with a therapeutic agent prior to installation around the ends of a lacerated tendon. Alternatively, the flexible tissue sheath 100 can be loaded with a therapeutic agent after installation around the ends of a lacerated tendon.

In this context, the terms “load” or “loading” means wetting, embedding, absorbing, adsorbing or otherwise introducing a therapeutic amount of the desired therapeutic agent onto or into the therapeutic agent carrier, with a “therapeutic amount” being a beneficial dosage based on clinical need.

The therapeutic agents can include drugs, cellular matters, biological factors, or a combination thereof. In a particular embodiment, the drugs can include antibiotics, analgesics, anti-inflammatory drugs, anti-TNF-alpha, steroids, anti-proliferative drugs, matrix metalloproteinase (MMP) inhibitors, or a combination thereof. Further, the cellular matters can include stem cell populations derived from bone marrow, stem cell populations derived from fat, other stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, platelet rich plasma (PRP), or a combination thereof.

In a particular embodiment, the flexible tissue sheath 100 can be coated with a hydrogel to prevent any adhesions of the tendon to the flexible tissue sheath 100. As such, after the tendon is healed, the flexible tissue sheath 100 can be removed. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

Description of a Flexible Tissue Sheath Installed Around a Lacerated Tendon

Referring now to FIG. 3, a lacerated tendon is shown and is generally designated 300. The lacerated tendon 300 can include a first end 304 and a second end 306. As shown in FIG. 3, a flexible tissue sheath, e.g., a flexible tissue sheath according to one or more embodiments described herein, can be installed around the lacerated tendon 300. For example, the flexible tissue sheath 100 show in FIG. 1 and FIG. 2 can be installed around the lacerated tendon 300.

In a particular embodiment, the first end 304 of the lacerated tendon 300 can be installed within the first end 104 of the body 102 of the flexible tissue sheath 100. Further, the second end 306 of the lacerated tendon 300 can be installed within the second end 106 of the body 102 of the flexible tissue sheath 100. The first end 304 of the lacerated tendon 300 can be attached, or otherwise retained, within the first end 104 of the body 102 of the flexible tissue sheath 100 by a first set of sutures 310. Also, the second end 306 of the lacerated tendon 300 can be attached, or otherwise affixed, within the second end 106 of the body 102 of the flexible tissue sheath 100 by a second set of sutures 312. Alternatively or in addition to sutures, one or both of the tendon ends can be retained in the body with a biocompatible adhesive. For example, the adhesive can include a surgical adhesive. The surgical adhesive can include an adhesive material containing bovine serum albumin and glutaraldehyde (aka, Bioglue), an adhesive material containing 2-octyl cyanoacrylate (aka, Dermabond), an adhesive material containing 2-butyl cyanoacrylate, an adhesive material containing fibrin, an adhesive material containing poly L-glutamic acid and gelatin, another tissue adhesive well known in the art, or a combination thereof. Accordingly, the flexible tissue sheath 100 can be used to couple the ends 304, 306 of the lacerated tendon 300 in order to allow the ends 304, 306 of the lacerated tendon 300 to heal.

It is to be understood that the flexible tissue sheath 100 can be used to repair fibrous connective tissue, e.g., tendons or ligaments. Further, methods of repairing a tendon or a ligament with a flexible tissue sheath according to one or more embodiments described herein can be substantially the same.

Description of a Method of Treating a Lacerated Tendon

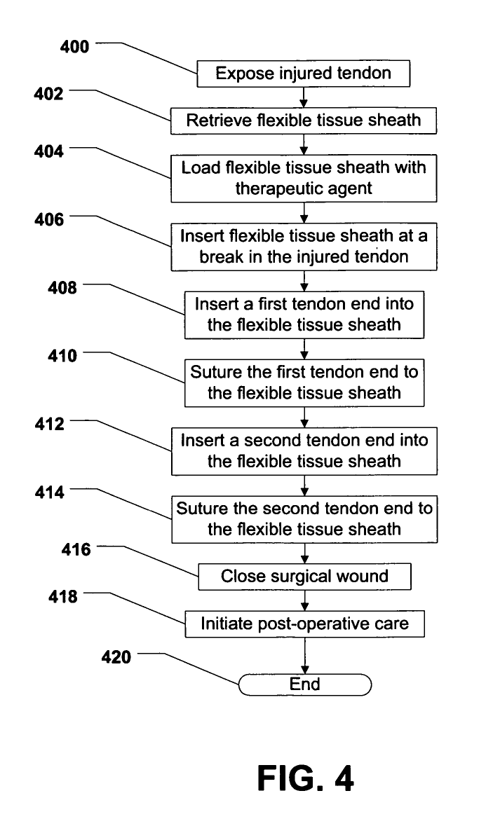

Referring now to FIG. 4, a method of treating a lacerated tendon is shown and commences at block 400. At block 400, the injured tendon can be exposed. Further, at block 402 a flexible tissue sheath, e.g., the flexible tissue sheath shown in FIG. 1 and FIG. 2, can be retrieved. Moving to block 404, the flexible tissue sheath can be loaded with one or more therapeutic agents.

The therapeutic agents can include drugs, cellular matters, biological factors, or a combination thereof. In a particular embodiment, the drugs can include antibiotics, analgesics, anti-inflammatory drugs, anti-TNF-alpha, steroids, anti-proliferative drugs, matrix metalloproteinase (MMP) inhibitors, or a combination thereof. Further, the cellular matters can include stem cell populations derived from bone marrow, stem cell populations derived from fat, other stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, platelet rich plasma (PRP), or a combination thereof.

Proceeding to block 406, the flexible tissue sheath can be installed at a break in the injured tendon. Moreover, at block 408, a first tendon end can be inserted into the flexible tissue sheath, e.g., into a first end of the flexible tissue sheath. At block 410, the first tendon end can be attached, e.g. sutured or the like, to the flexible tissue sheath. At block 412, a second tendon end can be inserted into the tissue sheath, e.g., into a second end of the flexible tissue sheath. Also, at block 414, the second tendon end can be attached, e.g. sutured or the like, to the tissue sheath.

Continuing to block 416, the surgical wound associated with exposing the injured tendon can be closed. The surgical wound can be closed by simply allowing the patient's skin to close due to the elasticity of the skin. Alternatively, the surgical wound can be closed using sutures, surgical staples, or any other suitable surgical technique well known in the art. At block 418, post-operative care can be initiated. The method then ends at state 420.

In a particular embodiment, as indicated in FIG. 4, the flexible tissue sheath can be loaded prior to placement around the lacerated tendon. However, in an alternative embodiment, the flexible tissue sheath can be loaded after placement around the lacerated tendon.

### Description of a Second Embodiment of a Flexible Tissue Sheath

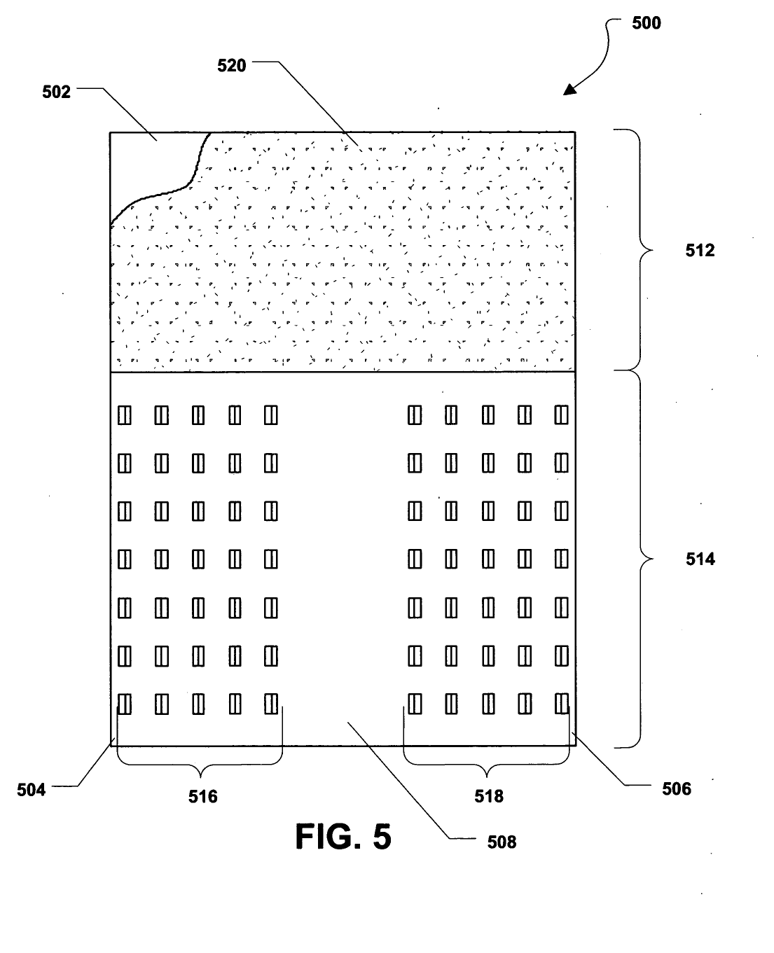

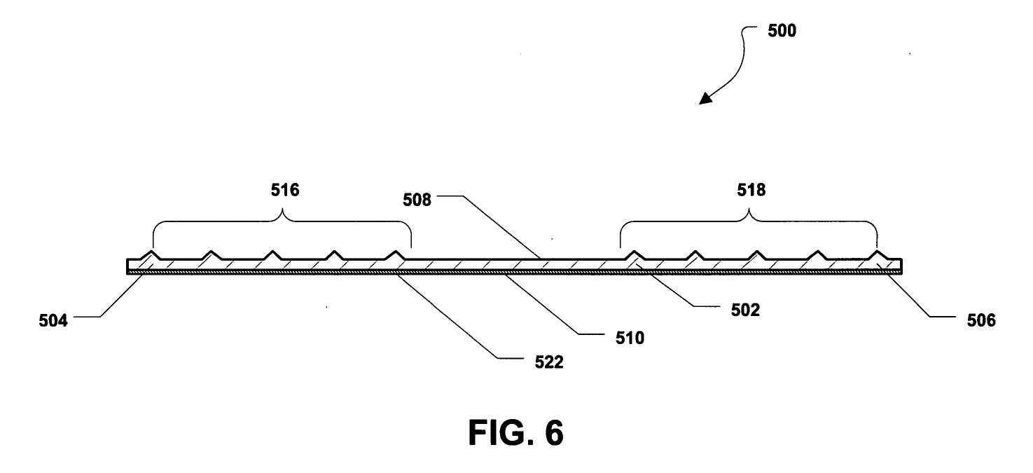

Referring to FIG. 5 and FIG. 6, a second embodiment of a flexible tissue sheath is shown and is generally designated 500. As illustrated, the flexible tissue sheath 500 includes a body 502. In a particular embodiment, the body 502 can be generally flat and generally rectangular. However, the body 502 can be wrapped around a tendon or ligament, as described in detail herein, to form a hollow, generally cylindrical structure. In a particular embodiment, the body 502 can be flexible. Also, the body 502 can have a tensile strength that can allow the flexible tissue sheath 500 to function in unison with a tendon around which the flexible tissue sheath 500 can be installed, as described herein. In a particular embodiment, the body 502 can define a first end 504 and a second end 506 that is opposite the first end 504. The body 502 can also include an interior surface 508 and an exterior surface 510.

As depicted in FIG. 5, the interior surface 508 can include a tissue engagement portion 514 and a flap portion 512. The tissue engagement portion 514 of the interior surface 508 can include a first set of tissue engagement structures 516 that can extend from the tissue engagement portion 514 of the interior surface 508 between a center of the body 502 and the first end 504 of the body 502. Further, the tissue engagement portion 514 of the interior surface 508 can include a second set of tissue engagement structures 518 that can extend from the tissue engagement portion 512 of the interior surface 508 between the center of the body 502 and the second end 506 of the body 502.

In a particular embodiment, the tissue engagement structures 516, 518 can be configured to engage tissue, e.g., a tendon or a ligament, when the flexible tissue sheath 500 is wrapped, or otherwise installed, around the tendon or ligament. Accordingly, the flexible tissue sheath 500 can be wrapped around an injured tendon, as described below. For example, the tissue engagement structures 516, 518 can engage the ends of a lacerated tendon to substantially prevent the ends of the lacerated tendon from being withdrawn from the flexible tissue sheath 500. In certain embodiments, such as when treating an intact tendon or ligament that has not been bisected, the tissue engagement structures 516, 518 can function to maintain the sheath 500 in a desired position on the tendon or ligament. The flexible tissue sheath can also function as a reinforcing member at a point of weakness (i.e., location of injury) along the tendon or ligament to maintain the integrity of the tissue while it heals.

In a particular embodiment, as illustrated in FIG. 5, the tissue engagement structures 516, 518 can be ramped structures, barbs, teeth, spikes, a combination thereof, or some other structure designed to engage tissue and prevent the tissue from sliding relative to the tissue engagement structures 516, 518. As such, in certain embodiments, two ends of a lacerated tendon can be placed adjacent to each other and the flexible tissue sheath 500 can be wrapped there around. Accordingly, the flexible tissue sheath 500 can act as a coupling mechanism in order to couple the ends of a lacerated tendon together to allow the ends of the lacerated tendon to heal and the tendon to repair itself.

As illustrated in FIG. 5, the flap portion 514 of the interior surface 508 can include an adhesive layer 520 that can be disposed thereon. As such, when the flexible tissue sheath 500 is wrapped around a tendon or ligament, as described herein, the adhesive layer 520 of the flap portion 514 can engage and adhere to a portion of the exterior surface 510, or a portion of a layer disposed thereon.

In a particular embodiment, the body 102 of the flexible tissue sheath 100 can be porous. As such, the flexible tissue sheath 100 can be loaded with a therapeutic agent prior to installation around the ends of a lacerated tendon. In this context, the terms “load” or “loading” means wetting, embedding, absorbing, adsorbing or otherwise introducing a therapeutic amount of the desired therapeutic agent onto or into the therapeutic agent carrier, with a “therapeutic amount” being a beneficial dosage based on clinical need.

The therapeutic agents can include drugs, cellular matters, biological factors, or a combination thereof. In a particular embodiment, the drugs can include antibiotics, analgesics, anti-inflammatory drugs, anti-TNF-alpha, steroids, anti-proliferative drugs, matrix metalloproteinase (MMP) inhibitors, or a combination thereof. Further, the cellular matters can include stem cell populations derived from bone marrow, stem cell populations derived from fat, other stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, platelet rich plasma (PRP), or a combination thereof.

FIG. 6 further indicates that a substantially impermeable layer 522 can be disposed on, or otherwise affixed to, the exterior surface 510 of the body 502. As such, the body 502 of the flexible tissue sheath 500 can be loaded with a therapeutic agent, as described herein, and the impermeable layer 522 can facilitate retention of the therapeutic agent within the body 502 of the flexible tissue sheath 500, which can further accelerated healing of the lacerated tendon.

In a particular embodiment, the impermeable layer 522 can be substantially smooth and can substantially prevent tissue adhesion to the body 502 of the flexible tissue sheath 500. Also, the impermeable layer 522 can substantially minimize scarring around the lacerated tendon and the flexible tissue sheath 500 used to repair the lacerated tendon.

Description of a Method of Treating an Injured Tendon

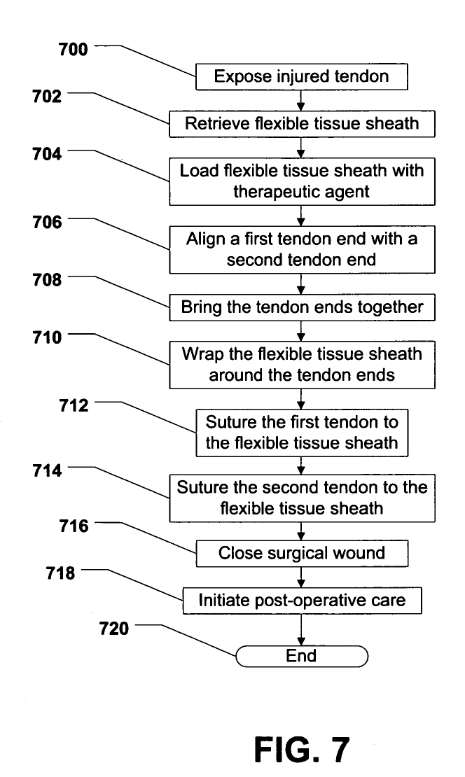

Referring to FIG. 7, a method of treating an injured tendon is shown and commences at block 700. At block 700, the injured tendon can be exposed. Further, at block 702 a flexible tissue sheath, e.g., the flexible tissue sheath shown in FIG. 5 and FIG. 6, can be retrieved. Moving to block 704, the flexible tissue sheath can be loaded with one or more therapeutic agents.

The therapeutic agents can include drugs, cellular matters, biological factors, or a combination thereof. In a particular embodiment, the drugs can include antibiotics, analgesics, anti-inflammatory drugs, anti-TNF-alpha, steroids, anti-proliferative drugs, matrix metalloproteinase (MMP) inhibitors, or a combination thereof. Further, the cellular matters can include stem cell populations derived from bone marrow, stem cell populations derived from fat, other stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, platelet rich plasma (PRP), or a combination thereof.

Continuing to block 706, when dealing with a lacerated tendon, a first tendon end can be aligned with a second tendon end. Also, at block 708, the tendon ends can be brought together. Thereafter, at block 710, the flexible tissue sheath can be wrapped around the tendon ends such that an adhesive layer contacts a surface of the sheath. At block 712, the first tendon end can be attached, e.g. sutured or the like, to the flexible tissue sheath. Moreover, at block 714, the second tendon end can be attached, e.g. sutured or the like, to the tissue sheath.

Continuing to block 716, the surgical wound associated with exposing the injured tendon can be closed. The surgical wound can be closed by simply allowing the patient's skin to close due to the elasticity of the skin. Alternatively, the surgical wound can be closed using sutures, surgical staples, or any other suitable surgical technique well known in the art. At block 718, post-operative care can be initiated. The method then ends at state 720.

### Description of a Third Embodiment of a Flexible Tissue Sheath

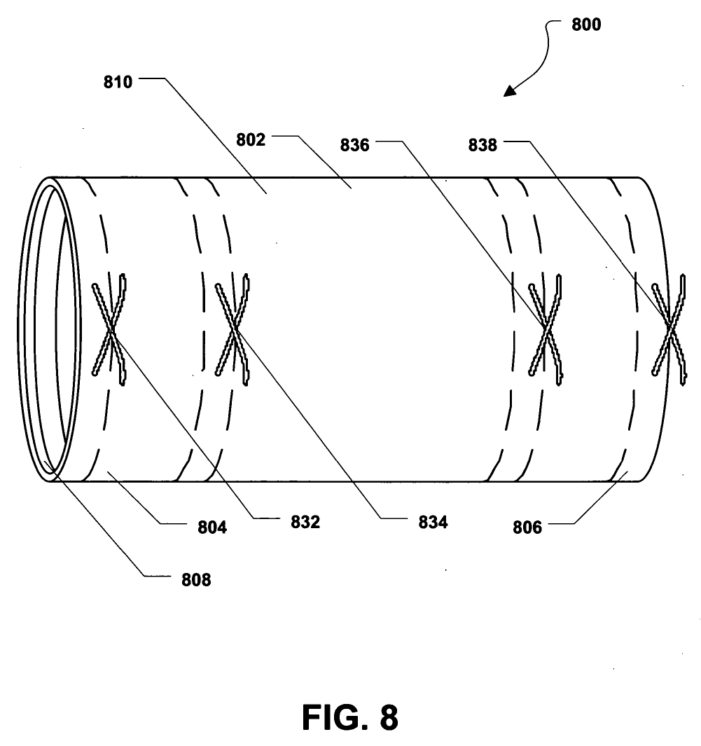

Referring to FIG. 8, a third embodiment of a flexible tissue sheath is shown and is generally designated 800. As illustrated, the flexible tissue sheath 800 includes a body 802. In a particular embodiment, the body 802 can be generally hollow and generally cylindrical. Further, the body 802 can be flexible. Also, the body 802 can have a tensile strength that can allow the flexible tissue sheath 800 to function in unison with fibrous connective tissue, e.g., a tendon or ligament, around which the flexible tissue sheath 800 can be installed, as described herein.

In a particular embodiment, body 802 of the flexible tissue sheath 800 can be made from a biocompatible material. Further, the biocompatible material can include a natural polymer, a synthetic polymer, a blend of a natural polymer and a synthetic polymer, or a combination thereof. The natural polymer can include collagen.

The synthetic polymers can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

The polymer blends can include a blend of collagen and one or more of the above synthetic polymers. Further, the polymer blends can include a blend of collagen and alginate, a blend of collagen and polyethylglycol (PEG), a blend of collagen and polylactone, or a combination thereof.

In a particular embodiment, the body 802 can define a first end 804 and a second end 806 that is opposite the first end 804. The body 802 can also include an interior surface 808 and an exterior surface 810.

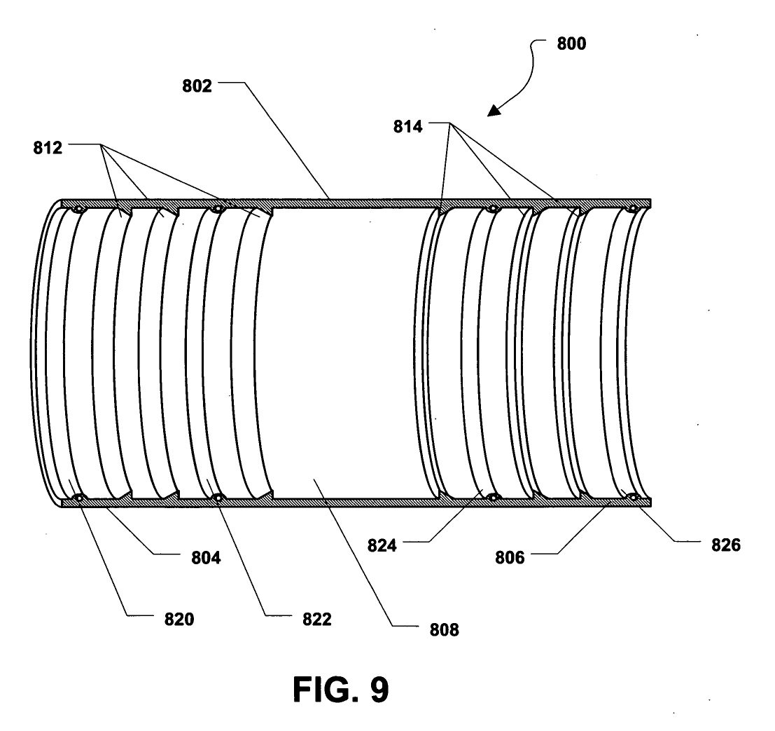

As depicted in FIG. 9, the interior surface 808 can include a first set of directional tissue engagement structures 812 that can extend from the interior surface 808 between a center of the body 802 and the first end 804 of the body 802. Further, the interior surface 808 can include a second set of directional tissue engagement structures 814 that can extend from the interior surface 808 between the center of the body 802 and the second end of 806 the body 802.

In a particular embodiment, the directional tissue engagement structures 812, 814 can be configured to engage tissue, e.g., a tendon or a ligament, in a single direction. Accordingly, the flexible tissue sheath 800 can be inserted over an end of a tendon without the directional tissue engagement structures 812, 814 interfering with the insertion of the flexible tissue sheath 800 over the end of the tendon. However, after the flexible tissue sheath 800 is inserted over the end of the tendon, the directional tissue engagement structures 812, 814 can engage the end of the tendon and substantially reduce the ease of removing the flexible tissue sheath 800 from the end of the tendon. Accordingly, it can be relatively easy to slide, or otherwise install, the flexible tissue sheath 800 over an end of a tendon and it can be relatively difficult to remove the flexible tissue sheath 800 from the end of the tendon.

In a particular embodiment, as illustrated in FIG. 9, the directional tissue engagement structures 812, 814 can be ramped structures, barbs, teeth, spikes, a combination thereof, or some other structure designed to allow tissue to slide relative to the directional tissue engagement structures 812 in a single direction. Further, in a particular embodiment, the first set of directional tissue engagement structures 812 can be oriented opposite the second set of directional tissue engagement structures 814.

In other words, each set of directional tissue engagement structures 812, 814 can each be oriented to allow a tendon end to be slid, or otherwise moved, into the flexible tissue sheath 800 from opposite ends 804, 806 of the sheath 800. As such, two ends of a lacerated tendon can be inserted into the flexible tissue sheath 800 and the flexible tissue sheath 800 can substantially prevent the ends of the lacerated tendon from being removed from the flexible tissue sheath 800. Accordingly, the flexible tissue sheath 800 can act as a coupling mechanism in order to couple the ends of a lacerated tendon together to allow the ends of the lacerated tendon to heal and the tendon to repair itself.

In various alternative embodiments, one or both sets of directional tissue engagement structures 812, 814 can include structures of various sizes. For example, the height of the structures can increase or decrease from the center of the body 802 to either or both ends 804, 806, thereby defining a tapered aperture through which the ligament or tendon is inserted or moved. Similarly, one or both sets of directional tissue engagement structures 812, 814 can vary in shape at one or more points along the longitudinal axis of the body 802 in order to accommodate tissue of various shapes and conditions.

FIG. 9 further shows that the interior surface 808 of the body 802 can be formed with a first draw string sleeve 820, a second draw string sleeve 822, a third draw string sleeve 824, and a fourth draw string sleeve 826. FIG. 8 indicates that a first drawstring 830 can be disposed within the first drawstring sleeve 820. A second drawstring 832 can be disposed within the second drawstring sleeve 822. A third drawstring 834 can be disposed within the third drawstring sleeve 824. Further, a fourth drawstring 836 can be disposed within the fourth drawstring sleeve 826. In a particular embodiment, the drawstrings 830, 832, 834, 836 can be tightened around a tendon in order to further prevent one or more ends of a lacerated tendon from withdrawing from the flexible tissue sheath 800.

As illustrated in FIG. 9, the exterior surface 810 of the body 802 can be substantially smooth. Further, the exterior surface 810 of the body 802 can substantially prevent tissue adhesion to the exterior surface 810 of the body 802. Also, the exterior surface 810 can substantially minimize scarring around the lacerated tendon and the flexible tissue sheath 800 used to repair the lacerated tendon.

In a particular embodiment, the body 802 of the flexible tissue sheath 800 can be porous. As such, the flexible tissue sheath 800 can be loaded with a therapeutic agent prior to installation around the ends of a lacerated tendon. Alternatively, the flexible tissue sheath 800 can be loaded with a therapeutic agent after installation around the ends of a lacerated tendon.

In this context, the terms “load” or “loading” means wetting, embedding, absorbing, adsorbing or otherwise introducing a therapeutic amount of the desired therapeutic agent onto or into the therapeutic agent carrier, with a “therapeutic amount” being a beneficial dosage based on clinical need.

The therapeutic agents can include drugs, cellular matters, biological factors, or a combination thereof. In a particular embodiment, the drugs can include antibiotics, analgesics, anti-inflammatory drugs, anti-TNF-alpha, steroids, anti-proliferative drugs, matrix metalloproteinase (MMP) inhibitors, or a combination thereof. Further, the cellular matters can include stem cell populations derived from bone marrow, stem cell populations derived from fat, other stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, platelet rich plasma (PRP), or a combination thereof.

In a particular embodiment, the flexible tissue sheath 800 can be coated with a hydrogel to prevent any adhesions of the tendon to the flexible tissue sheath 800. As such, after the tendon is healed, the flexible tissue sheath 800 can be removed. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

### Description of a Fourth Embodiment of a Flexible Tissue Sheath

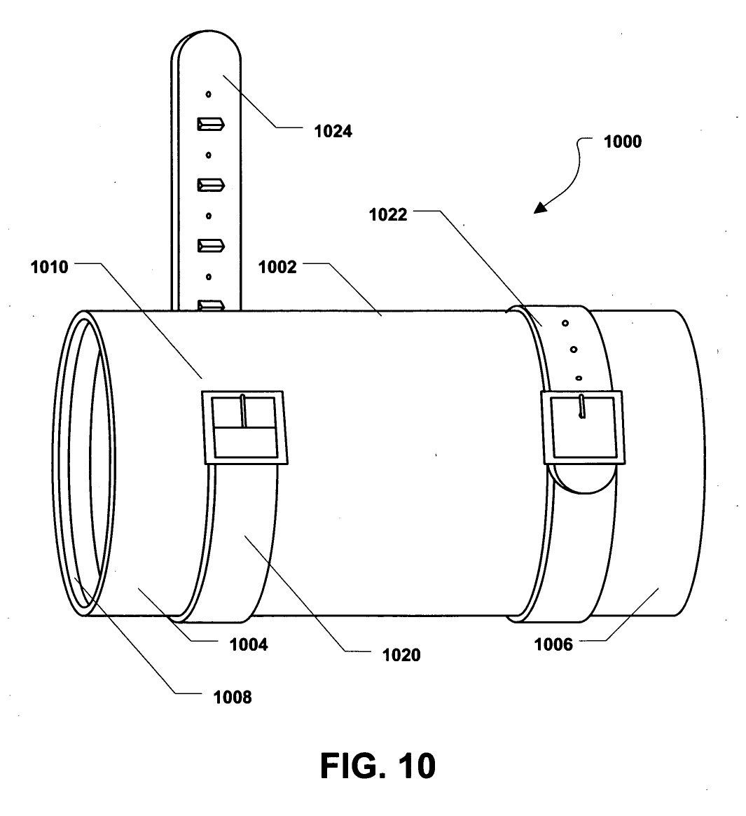

Referring to FIG. 10, a fourth embodiment of a flexible tissue sheath is shown and is generally designated 1000. As illustrated, the flexible tissue sheath 1000 includes a body 1002. In a particular embodiment, the body 1002 can be generally hollow and generally cylindrical. Further, the body 1002 can be flexible. Also, the body 1002 can have a tensile strength that can allow the flexible tissue sheath 1000 to function in unison with fibrous connective tissue, e.g., a tendon or ligament, around which the flexible tissue sheath 1000 can be installed, as described herein.

In a particular embodiment, body 1002 of the flexible tissue sheath 1000 can be made from a biocompatible material. Further, the biocompatible material can include a natural polymer, a synthetic polymer, a blend of a natural polymer and a synthetic polymer, or a combination thereof. The natural polymer can include collagen.

The synthetic polymers can include polyurethane materials, polyolefin materials, polyaryletherketone (PAEK) materials, silicone materials, or a combination thereof. Further, the polyolefin materials can include polypropylene, polyethylene, halogenated polyolefin, flouropolyolefin, or a combination thereof. The polyaryletherketone (PAEK) materials can include polyetherketone (PEK), polyetheretherketone (PEEK), polyetherketoneketone (PEKK), polyetherketoneetherketoneketone (PEKEKK), or a combination thereof.

The polymer blends can include a blend of collagen and one or more of the above synthetic polymers. Further, the polymer blends can include a blend of collagen and alginate, a blend of collagen and polyethylglycol (PEG), a blend of collagen and polylactone, or a combination thereof.

In a particular embodiment, the body 1002 can define a first end 1004 and a second end 1006 that is opposite the first end 1004. The body 1002 can also include an interior surface 1008 and an exterior surface 1010.

As depicted in FIG. 10, the interior surface 1008 can include a first set of directional tissue engagement structures 1012 that can extend from the interior surface 1008 between a center of the body 1002 and the first end 1004 of the body 1002. Further, the interior surface 1008 can include a second set of directional tissue engagement structures 1014 that can extend from the interior surface 1008 between the center of the body 1002 and the second end of 1006 the body 1002.

In a particular embodiment, the directional tissue engagement structures 1012, 1014 can be configured to engage tissue, e.g., a tendon or a ligament, in a single direction. Accordingly, the flexible tissue sheath 1000 can be inserted over an end of a tendon without the directional tissue engagement structures 1012, 1014 interfering with the insertion of the flexible tissue sheath 1000 over the end of the tendon. However, after the flexible tissue sheath 1000 is inserted over the end of the tendon, the directional tissue engagement structures 1012, 1014 can engage the end of the tendon and substantially reduce the ease of removing the flexible tissue sheath 1000 from the end of the tendon. Accordingly, it can be relatively easy to slide, or otherwise install, the flexible tissue sheath 1000 over an end of a tendon and it can be relatively difficult to remove the flexible tissue sheath 1000 from the end of the tendon.

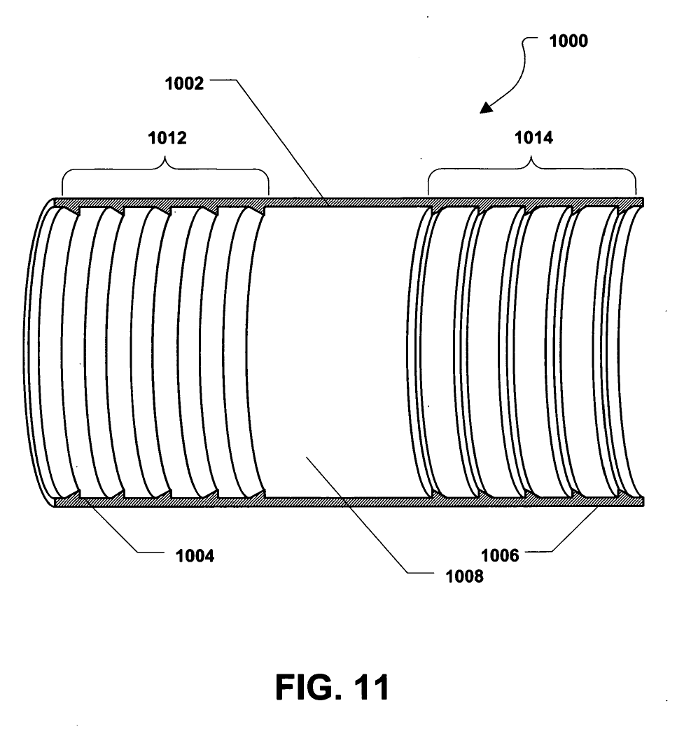

In a particular embodiment, as illustrated in FIG. 11, the directional tissue engagement structures 1012, 1014 can be ramped structures, barbs, teeth, spikes, a combination thereof, or some other structure designed to allow tissue to slide relative to the directional tissue engagement structures 1012 in a single direction. Further, in a particular embodiment, the first set of directional tissue engagement structures 1012 can be oriented opposite the second set of directional tissue engagement structures 1014.

In other words, each set of directional tissue engagement structures 1012, 1014 can each be oriented to allow a tendon end to be slid, or otherwise moved, into the flexible tissue sheath 1000 from opposite ends 1004, 1006 of the sheath 1000. As such, two ends of a lacerated tendon can be inserted into the flexible tissue sheath 1000 and the flexible tissue sheath 1000 can substantially prevent the ends of the lacerated tendon from being removed from the flexible tissue sheath 1000. Accordingly, the flexible tissue sheath 1000 can act as a coupling mechanism in order to couple the ends of a lacerated tendon together to allow the ends of the lacerated tendon to heal and the tendon to repair itself.

In various alternative embodiments, one or both sets of directional tissue engagement structures 1012, 1014 can include structures of various sizes. For example, the height of the structures can increase or decrease from the center of the body 1002 to either or both ends 1004, 1006, thereby defining a tapered aperture through which the ligament or tendon is inserted or moved. Similarly, one or both sets of directional tissue engagement structures 1012, 1014 can vary in shape at one or more points along the longitudinal axis of the body 1002 in order to accommodate tissue of various shapes and conditions.

FIG. 10 further shows that the flexible tissue sheath 1000 can include a first strap 1020 and a second strap 1022. Each strap 1020, 1022 can include a plurality of teeth 1024 that can extend through the flexible tissue sheath 1000 and engage an end of a tendon installed within the flexible tissue sheath 1000. In a particular embodiment, the straps 1020, 1022 can be tightened around a tendon in order to further prevent one or more ends of a lacerated tendon from withdrawing from the flexible tissue sheath 1000.

As illustrated in FIG. 10, the exterior surface 1010 of the body 1002 can be substantially smooth. Further, the exterior surface 1010 of the body 1002 can substantially prevent tissue adhesion to the exterior surface 1010 of the body 1002. Also, the exterior surface 1010 can substantially minimize scarring around the lacerated tendon and the flexible tissue sheath 1000 used to repair the lacerated tendon.

In a particular embodiment, the body 1002 of the flexible tissue sheath 1000 can be porous. As such, the flexible tissue sheath 1000 can be loaded with a therapeutic agent prior to installation around the ends of a lacerated tendon. Alternatively, the flexible tissue sheath 1000 can be loaded with a therapeutic agent after installation around the ends of a lacerated tendon.

In this context, the terms “load” or “loading” means wetting, embedding, absorbing, adsorbing or otherwise introducing a therapeutic amount of the desired therapeutic agent onto or into the therapeutic agent carrier, with a “therapeutic amount” being a beneficial dosage based on clinical need.

The therapeutic agents can include drugs, cellular matters, biological factors, or a combination thereof. In a particular embodiment, the drugs can include antibiotics, analgesics, anti-inflammatory drugs, anti-TNF-alpha, steroids, anti-proliferative drugs, matrix metalloproteinase (MMP) inhibitors, or a combination thereof. Further, the cellular matters can include stem cell populations derived from bone marrow, stem cell populations derived from fat, other stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, platelet rich plasma (PRP), or a combination thereof.

In a particular embodiment, the flexible tissue sheath 1000 can be coated with a hydrogel to prevent any adhesions of the tendon to the flexible tissue sheath 1000. As such, after the tendon is healed, the flexible tissue sheath 1000 can be removed. The hydrogels can include polyacrylamide (PAAM), poly-N-isopropylacrylamine (PNIPAM), polyvinyl methylether (PVM), polyvinyl alcohol (PVA), polyethyl hydroxyethyl cellulose, poly (2-ethyl) oxazoline, polyethyleneoxide (PEO), polyethylglycol (PEG), polyacrylacid (PAA), polyacrylonitrile (PAN), polyvinylacrylate (PVA), polyvinylpyrrolidone (PVP), or a combination thereof.

Description of a Second Method of Treating a Lacerated Tendon

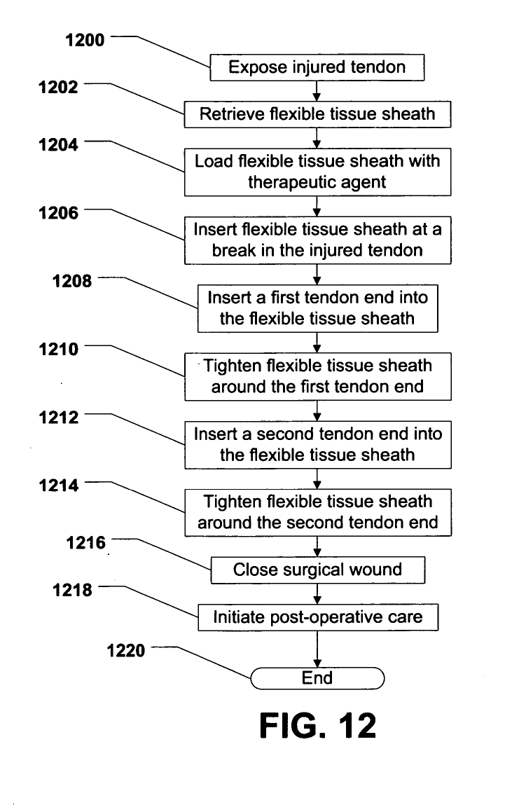

Referring now to FIG. 12, a second method of treating a lacerated tendon is shown and commences at block 1200. At block 1200, the injured tendon can be exposed. Further, at block 1202 a flexible tissue sheath, e.g., the flexible tissue sheath shown in FIG. 1 and FIG. 2, can be retrieved. Moving to block 1204, the flexible tissue sheath can be loaded with one or more therapeutic agents.

The therapeutic agents can include drugs, cellular matters, biological factors, or a combination thereof. In a particular embodiment, the drugs can include antibiotics, analgesics, anti-inflammatory drugs, anti-TNF-alpha, steroids, anti-proliferative drugs, matrix metalloproteinase (MMP) inhibitors, or a combination thereof. Further, the cellular matters can include stem cell populations derived from bone marrow, stem cell populations derived from fat, other stem cells, or a combination thereof. Also, the biological factor can include bone morphogenetic protein (BMP), cartilage-derived morphogenetic protein (CDMP), platelet derived growth factor (PDGF), insulin-like growth factor (IGF), LIM mineralization protein, fibroblast growth factor (FGF), osteoblast growth factor, platelet rich plasma (PRP), or a combination thereof.

Proceeding to block 1206, the flexible tissue sheath can be installed at a break in the injured tendon. Moreover, at block 1208, a first tendon end can be inserted into the flexible tissue sheath, e.g., into a first end of the flexible tissue sheath. At block 1210, the flexible tissue sheath can be tightened around the first tendon end. For example, a drawstring, a strap, or similar device, on the flexible tissue sheath can be tightened around the first tendon end. At block 1212, a second tendon end can be inserted into the tissue sheath, e.g., into a second end of the flexible tissue sheath. Also, at block 1214, the flexible tissue sheath can be tightened around the second tendon end. For example, a drawstring, a strap, or similar device, on the flexible tissue sheath can be tightened around the second tendon end.

Continuing to block 1216, the surgical wound associated with exposing the injured tendon can be closed. The surgical wound can be closed by simply allowing the patient's skin to close due to the elasticity of the skin. Alternatively, the surgical wound can be closed using sutures, surgical staples, or any other suitable surgical technique well known in the art. At block 1218, post-operative care can be initiated. The method then ends at state 1220.

In a particular embodiment, as indicated in FIG. 12, the flexible tissue sheath can be loaded prior to placement around the lacerated tendon. However, in an alternative embodiment, the flexible tissue sheath can be loaded after placement around the lacerated tendon.

### CONCLUSION

With the configuration of structure described above, the flexible tissue sheath provides a device that can be used to repair an injured tendon. The flexible tissue sheath includes one or more tissue engagement structures that can be configured to engage the ends of a lacerated tendon or ligament when the ends are inserted in the flexible tissue sheath or the flexible tissue sheath is wrapped around the ends. Alternatively, when dealing with an intact tendon or ligament that has not been bisected, the flexible tissue sheath can be wrapped around the injured area, as well as adjacent, healthy areas, to reinforce or stabilize the tendon or ligament while it heals. Further, the flexible tissue sheath can be porous and can be loaded with a therapeutic agent in order to facilitate healing of the lacerated tendon or ligament.

The above-disclosed subject matter is to be considered illustrative, and not restrictive, and the appended claims are intended to cover all such modifications, enhancements, and other embodiments that fall within the true spirit and scope of the present invention. Thus, to the maximum extent allowed by law, the scope of the present invention is to be determined by the broadest permissible interpretation of the following claims and their equivalents, and shall not be restricted or limited by the foregoing detailed description.

What is claimed is:

1. A flexible tissue sheath configured to treat a lacerated fibrous connective tissue, the flexible tissue sheath comprising:
a hollow body defining an interior surface and an exterior surface; and
at least one tissue engagement structure extending from the hollow body, wherein the tissue engagement structure is configured to engage an end of the lacerated fibrous connective tissue and substantially prevent the end of the lacerated fibrous connective tissue from withdrawing from the hollow body.

2. The flexible tissue sheath of claim 1, wherein the hollow body includes a first end and the flexible tissue sheath further comprises:
a first set of directional tissue engagement structures extending from the interior surface between a center of the body and the first end.

3. The flexible tissue sheath of claim 2, wherein the hollow body includes a second end and the flexible tissue sheath further comprises:
a second set of directional tissue engagement structures extending from the interior surface between the center of the body and the second end.

4. The flexible tissue sheath of claim 3, wherein the first set of directional tissue engagement structures is oriented substantially opposite the second set of directional tissue engagement structures.

5. The flexible tissue sheath of claim 4, wherein the first set of directional tissue engagement structures is configured to allow a first end of the lacerated fibrous connective tissue to be inserted into the hollow body and substantially prevent the first end of the lacerated fibrous connective tissue from withdrawing from the hollow body.

6. The flexible tissue sheath of claim 5, wherein the second set of directional tissue engagement structures is configured to allow a second end of the lacerated fibrous connective tissue to be inserted into the hollow body and substantially prevent the second end of the lacerated fibrous connective tissue from withdrawing from the hollow body.

7. The flexible tissue sheath of claim 1, wherein the body is porous and the flexible tissue sheath further comprises a therapeutic agent within the body.

8. The flexible tissue sheath of claim 7, wherein the therapeutic agent comprises a drug, a cellular matter, a biological factor, or a combination thereof.

9. The flexible tissue sheath of claim 8, wherein the drug comprises an antibiotic, an analgesic, an anti-inflammatory drug, an anti-TNF-alpha, a steroid, anti-proliferative drugs, matrix metalloproteinase (MMP) inhibitors, or a combination thereof.

10. The flexible tissue sheath of claim 9, wherein the cellular matter comprises bone marrow derived stem cells, lipo derived stem cells, or a combination thereof.

11. The flexible tissue sheath carrier of claim 10, wherein the biological factor comprises a bone morphogenetic protein (BMP), a cartilage-derived morphogenetic protein (CDMP), a platelet derived growth factor (PDGF), an insulin-like growth factor (IGF), an LIM mineralization protein, a fibroblast growth factor (FGF), an osteoblast growth factor, or a combination thereof.

12. The flexible tissue sheath of claim 1, further comprising a drawstring, wherein the drawstring can be tightened around the end of the lacerated fibrous connective tissue.

13. The flexible tissue sheath of claim 1, further comprising a strap, wherein the strap can be tightened around the end of the lacerated fibrous connective tissue.

14. The flexible tissue sheath of claim 13, wherein the strap comprises a tooth and the tooth engages the end of the lacerated fibrous connective tissue.

15. A method of treating a lacerated fibrous connective tissue, comprising:
inserting a first end of the lacerated fibrous connective tissue into a first end of a flexible tissue sheath so that the first end of the lacerated fibrous connective tissue engages a first set of tissue engagement structures.

16. The method of claim 15, further comprising:
inserting a second end of the lacerated fibrous connective tissue into a second end of a flexible tissue sheath so that the second end of the lacerated fibrous connective tissue engages a second set of tissue engagement structures.

17. The method of claim 16, further comprising:
attaching the first end of the lacerated fibrous connective tissue to the flexible tissue sheath.

18. The method of claim 17, further comprising:
attaching the first end of the lacerated fibrous connective tissue to the flexible tissue sheath.

19. The method of claim 15, further comprising:
loading the flexible tissue sheath with a therapeutic agent.

20. A flexible tissue sheath configured to treat an injured fibrous connective tissue, the flexible tissue sheath comprising:
a generally flat body configured to be wrapped around the injured fibrous connective tissue, wherein the body comprises:
a tissue engagement portion configured to engage a portion of the injured fibrous connective tissue when the flexible tissue sheath is wrapped around the injured fibrous connective tissue.

21. The flexible tissue sheath of claim 20, further comprising:
a first set of tissue engagement structures extending from the tissue engagement portion of the body.

22. The flexible tissue sheath of claim 21, wherein the first set of tissue engagement structures is configured to engage a first portion of the injured fibrous connective tissue and substantially prevent the first portion of the injured fibrous connective tissue from moving relative to the flexible tissue sheath when the flexible tissue sheath is wrapped around the injured fibrous connective tissue.

23. The flexible tissue sheath of claim 22, further comprising:
a second set of tissue engagement structures extending from the tissue engagement portion of the body.

24. The flexible tissue sheath of claim 23, wherein the second set of tissue engagement structures is configured to engage a second portion of the injured fibrous connective tissue and substantially prevent the second portion of the injured fibrous connective tissue from moving relative to the flexible tissue sheath when the flexible tissue sheath is wrapped around the injured fibrous connective tissue.

25. The flexible tissue sheath of claim 20, further comprising:
a flap portion adjacent to the tissue engagement portion, wherein the flap portion is configured to engage a portion of the flexible tissue sheath when the flexible tissue sheath is wrapped around the injured fibrous connective tissue.

26. The flexible tissue sheath of claim 25, further comprising:
an adhesive layer disposed on the flap portion.

27. The flexible tissue sheath of claim 20, wherein the body includes an exterior surface and the flexible tissue sheath further comprises:
a substantially impermeable layer disposed on the exterior surface of the body.

28. A method of treating an injured fibrous connective tissue, comprising:
positioning a flexible tissue sheath proximate to an injured area of the fibrous connective tissue, the flexible tissue sheath comprising a tissue engagement structure; and
wrapping the flexible tissue sheath around the fibrous connective tissue so that the tissue engagement structure engages the fibrous connective tissue proximate to the injured area.

29. The method of claim 28, further comprising:
loading the flexible tissue sheath with a therapeutic agent.

30. The method of claim 29, further comprising:
attaching the fibrous connective tissue to the flexible tissue sheath.

31. A method of treating a lacerated fibrous connective tissue, comprising:
inserting a first end of the lacerated fibrous connective tissue into a first end of a flexible tissue sheath so that the first end of the lacerated fibrous connective tissue engages a first set of tissue engagement structures; and
tightening the first end of the flexible tissue sheath around the first end of the lacerated fibrous connective tissue.

32. The method of claim 15, further comprising:
inserting a second end of the lacerated fibrous connective tissue into a second end of a flexible tissue sheath so that the second end of the lacerated fibrous connective tissue engages a second set of tissue engagement structures; and
tightening the second end of the flexible tissue sheath around the second end of the lacerated fibrous connective tissue.
